(19) United States
(12) Patent Application Publication
(10) Pub. No.: US 2008/0199457 A1
(43) Pub. Date: Aug. 21, 2008

(54) Anti-CD63 antibodies and methods of use thereof

(76) Inventors: Stefan Kraft, Brookline, MA (US); Jean-Pierre Kinet, Lexington, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/005,224

(22) Filed: Dec. 26, 2007

Related U.S. Application Data
(63) Continuation of application No. 11/332,890, filed on Jan. 17, 2006, now abandoned.
Application No. 11/332,890 is a continuation of application No. PCT/US2004/022035, filed on Jul. 8, 2004.
(60) Provisional application No. 60/487,525, filed on Jul. 14, 2003.

(51) Int. Cl. A61K 39/395; C07K 16/28; C07H 21/00; C07H 21/02

(52) U.S. Cl. 424/130.1; 530/387.1; 530/388.15; 536/23.1

(57) ABSTRACT

CD63 inhibition of IgE-mediated degranulation in mast cells and in vivo inhibition of allergic processes are described. The invention is drawn to methods of treating allergic conditions and anaphylaxis in a mammal comprising administering an effective amount of an agent that inhibits a function of CD63.

### RELATED APPLICATIONS

This application is a continuation of U.S. application Ser. No. 11/332,890, filed on Jan. 17, 2006, which is a continuation of International Application No. PCT/US2004/022035, which designated the United States, was filed on Jul. 8, 2004 and was published in English, and claims the benefit of U.S. Provisional Application No. 60/487,525, filed Jul. 14, 2003. The entire teachings of the above applications are incorporated herein by reference.

### BACKGROUND OF THE INVENTION

Immediate or Type 1 allergic reactions are largely attributed to IgE antibodies, although IgG antibodies can participate in and modulate allergic reactions. The allergy is generally caused by the activation of a subpopulation of immune cells, the mast cells and basophils. When antigen reacts with IgE antibody receptors on the cell's surface, the chemical mediators initiate the allergic reaction by acting on adjacent immune, epithelial, endothelial and smooth muscle cells and promote, in a longer term, the influx of other inflammatory and immune cells (neutrophils, eosinophils, monocytes, lymphocytes) into tissue. This influx of inflammatory cells predisposes the patient to recurrent and sometimes delayed, or prolonged allergic or hypersensitivity reactions. A distinction between immediate and delayed allergic reactions and delayed, chronic immune injury can also be made. The Type 1 allergic reactions are defined according to the location where they occur. For example, asthmatic reactions occur in the lungs, rhinitis in the nose, conjunctivitis in the eyes, systemic allergic reactions in the circulation and intestinal reactions in the gastrointestinal system.

Current therapeutic agents for treating allergy include, e.g., antihistamines and corticosteroids. However these therapeutic agents can have side effects, require frequent doses and/or treat only certain aspects of an allergic reaction. For example, antihistamines can cause drowsiness, sedation, gastrointestinal complications and/or dry mouth. Certain antihistamines can also produce adverse effects such as headaches, muscle spasms, angioedema, cardiac complications (in patients with QT syndrome or patients taking drugs that prolong the QT interval), myalgia and arthralgia. In addition, antihistamines may have to be taken at short intervals in order to maintain an effective level in patients. Antihistamines are also only designed to treat certain aspects of an allergic reaction (e.g., histamine release), rather than specifically targeting the cells responsible for production of histamine in an allergic reaction (e.g., mast cells, basophils).

Corticosteroids also have well documented side effects, particularly for long term use. For example, side effects of corticosteroid use include, e.g., iatrogenic Cushing syndrome, diabetogenic effects, alterations in electrolyte metabolism thereby potentially causing edema and/or hypertension, immunosuppression, osteoporosis, gastrointestinal ulcers, aseptic bone necrosis, adrenal gland insufficiency and many others.

Thus, what is needed are methods of inhibiting and/or decreasing the severity of allergy (e.g., immediate or Type 1 allergic reactions (e.g., hypersensitivity (anaphylactic) reactions)) and associated clinical conditions (e.g., asthma, rhinitis, anaphylactic shock).

### SUMMARY OF THE INVENTION

As described herein, antibodies to the tetraspanin CD63 (e.g., anti-CD63 mabs) have been isolated that inhibit FcεRI-mediated mast cell degranulation, but do not affect aggregation-dependent tyrosine phosphorylation or calcium mobilization. Importantly, as demonstrated for the first time by Applicants herein, CD63 antibodies inhibit mast cell degranulation in vivo, as measured by reduced passive cutaneous anaphylaxis responses in rats. These results show that anti-CD63 antibodies target a calcium-independent pathway of antigen receptor regulation that is accessible to engagement by membrane proteins and on which novel therapeutic approaches to allergic diseases can be based. In addition, the work described herein can also be used to develop model systems for the study of activation of mast cells through the FCER1 receptor and to improve the therapeutic capability to modulate the function of these cells.

In one embodiment, the invention is a method of treating (e.g., preventing, inhibiting or reducing the severity of) an allergic condition in a mammal, such as a human, comprising administering to the mammal an effective amount of an agent (e.g., an antibody or antigen-binding fragment thereof, an RNAi, an antisense oligonucleotide, a ribozyme, a small organic molecule, a peptide, a peptidomimetic) that inhibits a function of CD63 (e.g., binding to an integrin (e.g., β1-integrin) or other binding partner of CD63). In particular embodiments, the allergic condition is selected from the group consisting of asthma, rhinitis, hay fever, conjunctivitis, atopic dermatitis, atopic eczema, systemic allergic reactions, intestinal reactions of the gastrointestinal system, inflammatory diseases and autoimmune diseases (e.g., multiple sclerosis, rheumatoid arthritis, mastocytosis).

In another embodiment, the invention is a method of inhibiting passive cutaneous anaphylaxis in a mammal comprising administering to the mammal an effective amount of an agent that inhibits a function of CD63.

In another embodiment, the invention is a method of inhibiting anaphylaxis in a mammal (e.g., human) comprising administering to the mammal an effective amount of an agent that inhibits a function of CD63.

In another embodiment, the invention is a method of treating an allergic condition in a mammal comprising administering to the mammal an effective amount of an agent (e.g., an agent that binds to a tetraspanin (e.g., CD63)) that inhibits the interaction of the tetraspanin with an integrin or other partner molecule (e.g., ProHB-EGF, CD9P-1, tetraspanin (e.g., CD81, CD9)).

In another embodiment, the invention is an assay for identifying an agent that alters the interaction of CD63 and a binding partner of CD63 (e.g., integrin (e.g., β1-integrin), ProHB-EGF, CD9P-1, tetraspanin (e.g., CD81, CD9)). In the assay, a cell bearing CD63 (e.g., cell-surface CD63), a binding partner of CD63 and an agent to be tested are combined under conditions suitable for binding of CD63 to the binding partner and the level of CD63-mediated signal transduction is determined, wherein if the level of CD63-mediated signal transduction is altered relative to a control, the agent alters CD63-mediated signal transduction.

In another embodiment, the invention is a human antibody that binds CD63 (e.g., human CD63). In yet another embodiment, the invention is an agent (e.g., an RNAi molecule, an antisense oligonucleotide, a ribozyme), wherein the agent is an inhibitor of a function of CD63. In still another embodiment, the invention is an antibody that binds CD63 and can inhibit degranulation at a concentration of 10 μg/ml or less.

### BRIEF DESCRIPTION OF THE DRAWINGS

FIG. 1 is a set of histograms illustrating that DNP-HSA induces IgE-mediated degranulation and that this FcεR1-induced degranulation is inhibited by anti-CD63 mAb 7A6. RBL-2H3 cells were preincubated with either 1, 2, 4 or 8 μg/ml of isotype control antibody (IgG1) or anti-CD63 mAb 7A6, prior to triggering of FcεR1 with 100 ng/ml of DNP-HSA. Data are expressed as mean cpm.

FIG. 2 is a set of histograms illustrating that PMA/ionomycin induces degranulation and that this degranulation is not inhibited by anti-CD63 mAb 7A6. RBL-2H3 cells were preincubated with either 1, 2, 4 or 8 μg/ml of isotype control antibody (IgG1) or anti-CD63 mAb 7A6, prior to triggering of FcεR1 with 20 nM of PMA+2 μM of ionomycin. Data are expressed as mean cpm.

FIG. 3 is a histogram showing inhibition of passive cutaneous anaphylaxis in CD rats by anti-CD63 mAb 7A6 and anti-CD81 mAb 5D1. Female CD rats were injected with 5, 10 or 25 ng of anti-DNP IgE (labeled “IgE [ng]”) mixed with 50 μg of control antibody (labeled “IgG1”), 50 μg of anti-CD63 mAb 7A6 (labeled “7A6”), or 50 μg of anti-CD81 mAb 5D1 (labeled “5D1”; see U.S. Pat. No. 6,423,501). Data are expressed as μg of Evan's Blue (EB) dye extracted, based on a standard curve of dilutions of Evan's Blue in formamide.

FIG. 4 is another histogram showing inhibition of passive cutaneous anaphylaxis in CD rats by anti-CD63 mAb 12A10 and anti-CD81 mAb 5D1. Female CD rats were injected with 5, 10 or 25 ng of anti-DNP IgE (labeled “αDNP-IgE”) mixed with 50 μg of control antibody (labeled “IgG1”), 50 μg of anti-CD63 mAb 12A10 (labeled “αCD63”), or 50 μg of anti-CD81 mAb 5D1 (labeled “αCD81”). Data are expressed as % amount EB extraction (i.e., the percentage of μg EB extracted from mAb 12A10+IgE injections versus μg EB extracted from IgG1+IgE).

FIG. 5 is a histogram illustrating that anti-CD63 mAb 12A10 and anti-CD81 mAb 5D1 inhibit adhesion of RBL-2H3 cells to fibronectin. 96-well tissue culture microplates were coated with 10 μg/ml fibronectin, starved in culture medium containing no FCS, detached with trypsin and resuspended at 1×10⁶ cells/ml in culture medium containing 0.1% BSA and no FCS. Control antibodies (labeled “IgG1”) or inhibitory antibodies against the tetraspanins CD63 (labeled “CD63”) and/or CD81 (labeled “CD81”) or β1-integrins (labeled “α5+β1”) were added at 10, 20 or 40 μg/ml for 1 hour at 37° C. Data are expressed as absorption of incorporated dye at 570 nm. b1=no RBL-2H3 cells added; bg=no fibronectin in wells; max=RBL-2H3 cells added but no BSA blocking step; nil=no antibody added.

FIG. 6 is a histogram illustrating that anti-CD63 mAb 12A10 and anti-CD81 mAb 5D1 do not inhibit adhesion of RBL-2H3 cells to laminin-1. 96-well tissue culture microplates were coated with 20 μg/ml laminin-1, starved in culture medium containing no FCS, detached with trypsin and resuspended at 1×10⁶ cells/ml in culture medium containing 0.1% BSA and no FCS. Control antibodies (labeled “IgG1”) or inhibitory antibodies against the tetraspanins CD63 (labeled “CD63”) and/or CD81 (labeled “CD81”) or β-integrins (labeled “β1”) were added at 10, 20 or 40 μg/ml for 1 hour at 37° C. Data are expressed as absorption of incorporated dye at 570 nm. b1=no RBL-2H3 cells added; bg=no fibronectin in wells; max=RBL-2H3 cells added but no BSA blocking step; nil=no antibody added.

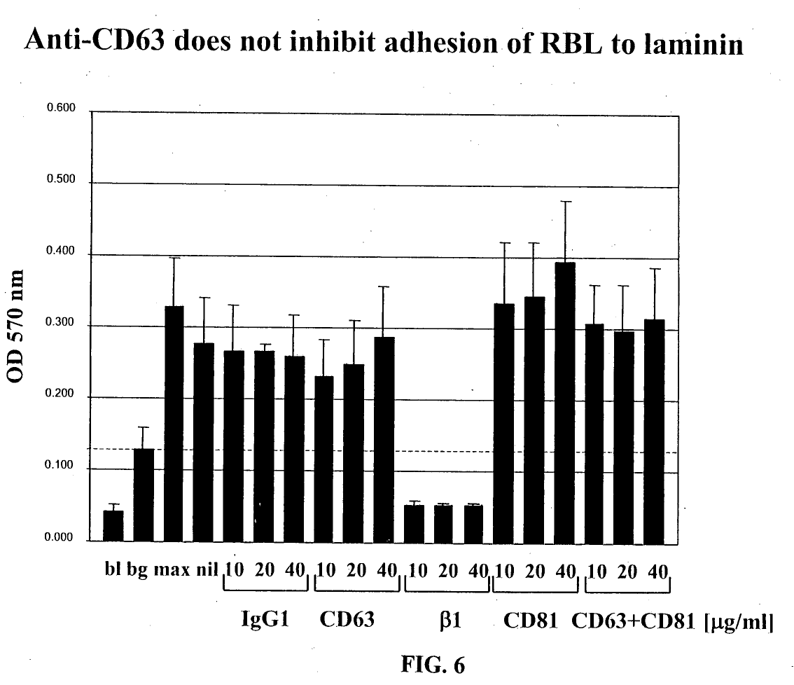

FIG. 7 depicts a nucleotide sequence of a DNA sequence encoding human (Homo sapiens) CD63 (SEQ ID NO:1; GenBank Accession No. NM_{—}001780).

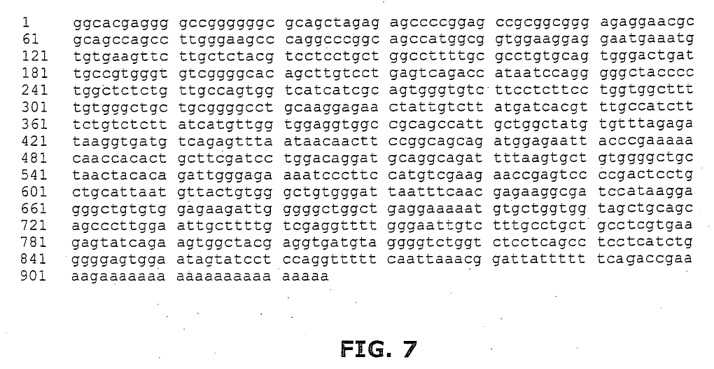

FIG. 8 depicts a human (Homo sapiens) CD63 amino acid sequence (SEQ ID NO:2; GenBank Accession No. NP_{—}001171).

### DETAILED DESCRIPTION OF THE INVENTION

A description of preferred embodiments of the invention follows.

Mast cells are important effector cells in IgE-dependent immune responses and allergic diseases (Galli, New. Engl. J. Med. 328:257-265 (1993)), and mast cells also contribute to host defense against parasites and bacteria (Echtenacher et al., Nature 381:75-77 (1996); Galli and Wershil, Nature 381:21-22 (1996)). Crosslinking of FcεRI-IgE complexes on mast cells and basophils by multivalent antigen initiates a signaling cascade characterized by tyrosine kinase activation, calcium release and influx and, later, by degranulation and release of inflammatory mediators (Kinet, J. P., Annu. Rev. Immunol. 17:931-972 (1999); Turner, H., et al., Nature 402 (6760 Suppl.):B24-B30 (1999)).

Like the B and T cell antigen receptors, FcεRI lacks endogenous signaling capacity and utilizes tyrosine phosphorylation to recruit signaling effector molecules. Receptor aggregation leads to phosphorylation and/or activation of several protein tyrosine kinases (PTKs), such as Lyn, Fyn, Syk, Btk, Itk, Fer, and FAK (Jouvin et al., J. Biol. Chem. 269:5918-5925 (1994); Penhallow et al., J. Biol. Chem. 270:23362-23365 (1995); Scharenberg et al., EMBO J. 14:3385-3394 (1995); Kawakami et al., Mol. Cell. Biol. 14:5108-5113 (1994); Kawakami et al., J. Immunol. 155:3556-3562 (1995); Hamawy et al., J. Biol. Chem. 268:6851-6854 (1993)), as well as protein kinase C isoenzymes (Ozawa et al., J. Biol. Chem. 268:1749-1756 (1993)), MAP kinase (Hirasawa et al., J. Biol. Chem. 270:10960-10967 (1995)), PI3-kinases, and other signaling molecules, such as Cbl and the adaptors Shc, Gab2 and LAT (Ota et al., J. Exp. Med. 184:1713-1723 (1996); Jabril-Cuenod et al., J. Biol. Chem. 271:16268-16272 (1996); Kinet, J. P., Annu. Rev. Immunol. 17:931-972 (1999); Turner, H., et al., Nature 402 (6760 Suppl.):B24-B30 (1999)).

The precise role of many of these proteins in degranulation remains undefined. However, it is clear that FcεRI-mediated calcium mobilization, degranulation, and letikotriene and cytokine synthesis depend on early tyrosine kinase activation events, involving Lyn, Fyn and Syk kinases. FcεRI signaling is initiated by tyrosine phosphorylation of immunoreceptor tyrosine-based activation motifs (ITAM; defined by the sequence (D/E)X₂YX₂LX₆₋₇YX₂(LI) (Flaswinkel et al., Semin. Immunol 7:21-27 (1995)). Phosphorylated ITAMs (pITAMs) facilitate binding of SH2-domain-containing proteins to FcεRI (Johnson et al., J. Immunol. 155:4596-4603 (1995); Kimura et al., J. Biol. Chem. 271:27962-27968 (1996)). Currently, it is believed that degranulation of mast cells is initiated by a membrane-localized signaling complex that is organized by the Gab2 adaptor protein inducing a calcium-independent pathway and another signaling complex, organized by the adaptor LAT, which induces a calcium-dependent pathway (Parravicini, V., et al., Nat. Immunol. 3(8):741-728 (2002)).

In addition to activation events, receptor-activated PTKs initiate the regulation of antigen receptor signaling by phosphorylating tyrosine-based motifs on membrane receptors known as inhibitory receptors (Scharenberg and Kinet, Cell 87:961-964 (1996); Cambier, Proc. Natl. Acad. Sci. USA 94:5993-5995 (1997)). These proteins bind SH2-domain-containing phosphatases, the tyrosine phosphatases SHP-1 and SHP-2 and the phosphatidylinositol (3, 4, 5) 5′ phosphatase SHIP, upon coengagement with antigen or growth factor receptors. Although the molecular targets are still being defined, phosphatase recruitment to inhibitory receptors has one of two general effects on signaling. Engagement of inhibitory receptors that preferentially bind SHIP, such as the low affinity receptor for IgG (FcγRIIb1) (Ono et al., Nature 383:263-266 (1996)), results in selective inhibition of calcium influx with little or no effect on receptor-mediated calcium release or tyrosine phosphorylation. On the other hand, killer cell inhibitory receptors (KIR) bind SHP-1 upon receptor costimulation, resulting in reduced tyrosine phosphorylation, calcium release from the ER and calcium influx (Burshtyn et al., Immunity 4:77-85 (1996); Binstadt et al., Immunity 5:629-638 (1996)). In both mechanisms, calcium mobilization is inhibited along with downstream signaling events.

IgE-dependent activation of mast cells primarily occurs through antigen-mediated crosslinking of IgE-FcεRI complexes that initiate a signaling cascade ultimately leading to release of proinflammatory mediators (Scharenberg and Kinet, Chem. Immunol. 61:72-87 (1995)). FcεRI is a member of the multi-subunit, antigen receptor family that includes B and T cell receptors (BCR and TCR) and receptors for the Fc portions of IgA and IgG (Ravetch and Kinet, Ann. Rev. Immunol. 9:457-492 (1991)); Kinet, J. P., Annu. Rev. Immunol. 17:931-972 (1999); Turner, H., et al., Nature 402 (6760 Suppl.):B24-B30 (1999)). These receptors share common features of immunoglobulin-like ligand binding subunit(s) and associated signaling polypeptides that lack endogenous enzymatic activity.

In mast cells, both FcεRI and FcγRIII are expressed as αβγg_{γ2} tetramers in which the respective β and FcRγ signaling chains are identical and the ligand-binding α chains are different. In FcεRI, the high affinity IgE binding domain is localized to the FcεRIα subunit (Blank et al., J. Biol. Chem. 266:2639-2646 (1991)) and IgE binding to FcεRIα itself does not contribute to signaling. The FcεRIP chain and the FcRγ homodimer are the signaling components of the FcεRI (αβγ2) tetrameric receptor. Both FcεRIβ and FcRγ have one copy per chain of the immunoreceptor tyrosine-based activation motif (ITAM; Flaswinkel et al., Semin. Immunol. 7:21-27 (1995), Cambier, J. Immunol. 155:3281-3285 (1995)).

FcεRI signaling is an aggregation-dependent phenomenon in which multivalent antigen crosslinking of IgE-FcεRI complexes initiates a signaling cascade that is mediated by cytoplasmic tail motifs known as immunoreceptor tyrosine based activation motifs (ITAMs) that become phosphorylated by src family kinases (Shaw et al., Semin. Immunol. 7:13-20 (1995)). Signaling through FcεRI is characterized initially by tyrosine phosphorylation of FcεRIP and FcRγ ITAMs by the β-associated src family kinase lyn (Jouvin et al., J. Biol. Chem. 269:5918-5925 (1994)). The lyn-phosphorylated ITAM (PITAM) interaction results in lyn activation. Direct binding of lyn to fusion proteins containing the FcεRIβ, but not the FcRγ ITAM, has been demonstrated (Jouvin et al., J. Biol. Chem. 269:5918-5925 (1994)). pITAM peptides have been shown to induce lyn phosphorylation both in permeabilized cells and in vitro (Johnson et al., J. Immunol. 155:4596-4603 (1995)).

Following lyn activation, syk is recruited to FcRγ pITAMs via its SH2 domains, where it is phosphorylated and activated (Scharenberg and Kinet, Chem. Immunol. 61:72-87 (1995); Jouvin et al., J. Biol. Chem. 269:5918-5925 (1994)). FcRγ pITAM peptides were much more effective than FcεRIP pITAM peptides at activating syk in vitro in unstimulated RBL-2H3 lysates (Shiue et al., J. Biol. Chem. 270:10498-10502 (1995)). Activated lyn and syk phosphorylate a number of intracellular substrates, including PLCγ1, BTK, ITK and cbl (Rawlings et al., Science 271:822-825 (1996); Kawakami et al., J. Immunol. 155:3556-3562 (1995)), and ultimately membrane-localized signaling complexes, organized by the adaptors LAT and Gab2, are formed (Parravicini, V., et al., Nat. Immunol. 3(8):741-728 (2002)). Following initial tyrosine kinase activation events, FcεRI signaling, like that of other antigen receptors, involves calcium release from the endoplasmic reticulum (tyrosine kinase-dependent) and a calcium influx, both of which precede degranulation and the release of preformed mediators by granule fusion with the cytoplasmic membrane. In addition, calcium mobilization through FcεRI appears to utilize sphingosine kinase and sphingosine-1-phosphate (S-1-P) (Choi et al., Nature 380:634-636 (1996)), as opposed to the classical phospholipase C/InsP3 pathway.

The rat basophilic leukemia cell line, RBL-2H3, has been widely employed as a model cell in the study of FcεRI-mediated activation. There have been a few reports of monoclonal antibodies (mAbs) directed to membrane components in which co-ligation inhibits FcεRI-mediated degranulation in mast cells. The best characterized examples are MAFA (mast cell function-associated antigen) (Guthmann et al., Proc. Natl. Acad. Sci. USA 92:9397-9401 (1995)), FcγRIIB (Malbec, O., et al., J. Immunol 160(4):1647-1658 (1998)) gp49b1 (Katz et al., Proc. Natl. Acad. Sci. USA 93:10809-10814 (1996)), and the tetraspanin, CD81 (U.S. Pat. No. 6,423,501).

MAFA is a 20 kd C-type lectin expressed in RBL-2H3 cells both as a monomer and disulphide-linked homodimer that inhibits degranulation by acting upstream of FcεRI-mediated activation of phospholipase Cgl activation by tyrosine kinases (Guthmann et al., Proc. Natl. Acad. Sci. USA 92:9397-9401 (1995)). MAFA, FcγRIIB and gp49BI form classical inhibitory receptors that are capable of abrogating antigen receptor signaling by the binding and activation of phosphatases to inhibitory ITIM (immunoreceptor tyrosine-based inhibitory motifs) within their cytoplasmic tails (Vivier, E., et al., Immunol. Today 18(6):286-291 (1997)). In contrast, degranulation can be inhibited by antibodies against the tetraspanin, CD81, but this requires no coligation and CD81 shows no ITIMs (U.S. Pat. No. 6,423,501, the entire teachings of which are incorporated herein by reference). CD81 belongs to the family of proteins known as tetraspanins (also known as tetraspans or the transmembrane 4 superfamily (TM4SF)). Tetraspanins are characterized by 4 transmembrane domains, and can form a network of multimolecular complexes, the ‘tetraspanin web’, in which integrins are included (Boucheix, C., et al., Cell Mol. Life. Sci. 58:1189-1205 (2001)). It has been shown that anti-CD81 antibodies can inhibit IgE-mediated degranulation in mast cells in a calcium-independent manner (U.S. Pat. No. 6,423,501).

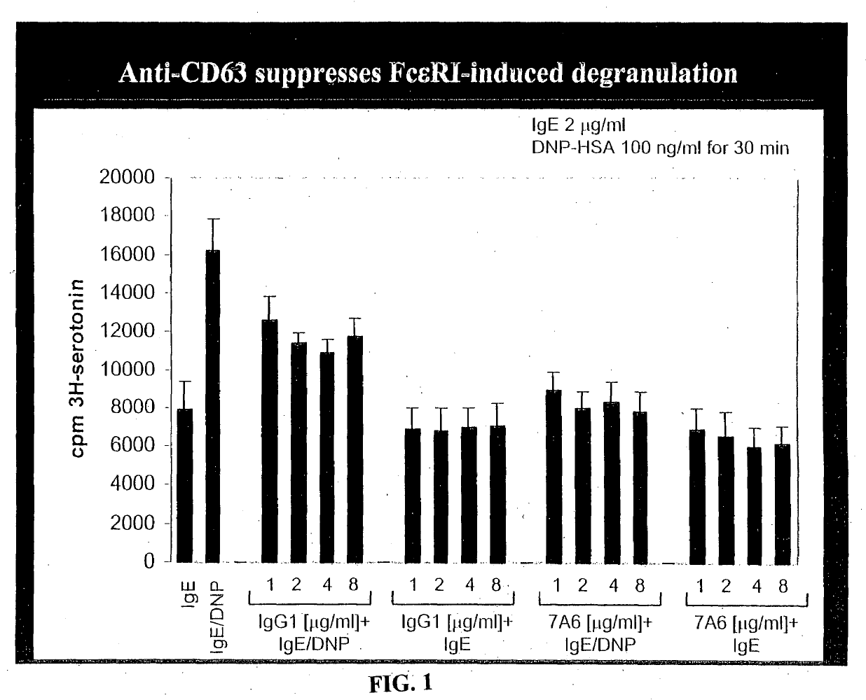

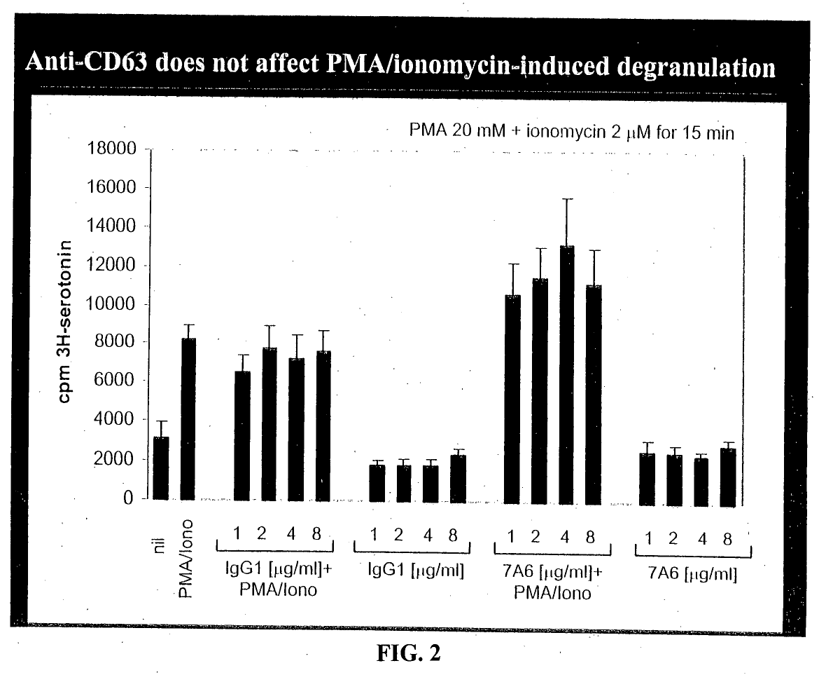

In addition, antibodies to the glycolipid Gd1b and the AD1 antigen (rat homologue of CD63) have also been described to inhibit FcεRI-mediated degranulation in RBL-2H3 cells (Kitani, S., et al., J. Biol. Chem., 266(3):1903-1909 (1991)). Kitani et al. describe a monoclonal antibody, mAb AD1, which has low affinity for AD1 (rat homologue of CD63; K_{d} of 4.6×10⁻⁸ M/l when tested for binding to RBL-2H3 cells) (Kitani et al., id., see FIG. 2). Kitani et al. further report that mAb AD1 was able to inhibit IgE-mediated histamine release when assayed in RBL-2H3 cells (Kitani et al., id.; see FIG. 1, maximum inhibition of 49±10% with 100 μg/ml mAb AD1). Importantly, Kitani et al. do not provide any in vivo data on mAb AD1.

Clustering of the high affinity IgE receptor (FcεRI) by antigen initiates a signaling cascade characterized by tyrosine kinase activation, calcium release and influx and, later, by degranulation and release of inflammatory mediators. In order to find negative regulators of FcεRI, a panel of monoclonal antibodies was produced by injecting BALB/c mice with RBL-2H3 cells and the resulting hybridoma supernatants were screened for their ability to inhibit serotonin release upon high affinity IgE receptor (FcεRI) triggering on RBL-2H3 (a standard assay for mast cell degranulation). Three monoclonal antibodies, designated mAb 7A6, mAb 12A2 and mAb 12A10, inhibited serotonin release and immunoprecipitated a 50-60 kD protein from RBL-2H3 cells. As described herein, it was shown that the 50-60 kD protein recognized by these antibodies was rat CD63 and that these anti-CD63 antibodies inhibited FcεR1-mediated mast cell degranulation and passive cutaneous anaphylaxis.

Signaling through the high affinity receptor for immunoglobulin E (FcεRI) results in the coordinate activation of tyrosine kinases prior to calcium mobilization. Receptors capable of interfering with the signaling of antigen receptors, such as FcεRI, recruit tyrosine and inositol phosphatases that results in diminished calcium mobilization. As described herein, anti-CD63 antibodies inhibit FcεRI-mediated mast cell degranulation, but, surprisingly, do not affect aggregation-dependent tyrosine phosphorylation or calcium mobilization. Importantly, as demonstrated for the first time herein, CD63 antibodies inhibit mast cell degranulation in vivo, as measured by reduced passive cutaneous anaphylaxis responses in rats. These results show that anti-CD63 antibodies target a calcium-independent pathway of antigen receptor regulation that is accessible to engagement by membrane proteins and on which novel therapeutic approaches to allergic diseases can be based. In addition, the work described herein can also be used to develop model systems for the study of activation of mast cells through the FcεRI receptor and to improve the therapeutic capability to modulate the function of these cells.

CD63, also known as LIMP, MLA1, PTLGP40, gp55, granulophysin, LAMP-3, ME491 and NGA (www.ncbi.nlm.nih.gov/prow/guide/1469534145_g.htm) is a member of the family of proteins known as tetraspanins. As described above, tetraspanins are characterized by 4 transmembrane domains which delimit two extracytoplasmic regions of unequal sizes, a small extracellular loop (EC1) containing 20-28 amino acids and a large extracellular loop (EC2) containing 76-131 amino acids (Boucheix, C., et al., Cell Mol. Life. Sci. 58:1189-1205 (2001)). At least 26 tetraspanin family members have been identified, including, for example, CD9, CD37, CD53, CD63, CD81/TAPA-1, CD82, CD151, C0-029, NAG-2, Oculospanin, CD231, SAS, Uroplakin 1b, Uroplakin 1a and Peripherin/RDS (Boucheix, C., et al., Cell Mol. Life Sci. 58:1189-1205 (2001); Wright and Tomlinson, Immunol. Today 15:588-594 (1994)). Although the function of tetraspanins is not precisely known, it appears that they play a major role in membrane biology and can modulate the signaling of other membrane receptors (see, e.g., U.S. Pat. No. 6,423,501). In addition, tetraspanins can form a network of multimolecular complexes, the ‘tetraspanin web’, in which integrins are included (Boucheix, C., et al., Cell Mol. Life. Sci. 58:1189-1205 (2001)). For example, CD63 can associate with various integrins, including α3β1, α6β1, Integrin α_{L}β₂, as well as ProHB-EGF and CD9P-1 and other tetraspanins (e.g., CD81, CD9) (Boucheix, C., et al., Cell Mol. Life Sci. 58:1189-1205 (2001); the entire teachings of which are incorporated herein by reference).

CD63, which has a lysosome-targeting signal, is expressed in a number of tissues and cell types. For example, CD63 has been shown to be strongly expressed in platelet lysosomes (Azorsa, D., et al., Blood 78:280-284 (1991)), in Weibel-Palade bodies of endothelial cells (Vischer, U. M., et al., Blood 82:1184-1191 (1993)) and in the azurophil granules of neutrophil granulocytes (Fitter, S., et al., Biochem J. 338:61-70 (1999)). Additional studies have demonstrated that CD63 is found in major histocompatibility (MHC) class II-enriched compartments (MIIC) of B lymphocytes (Escola, J. M., et al., J. Biol. Chem. 273:20121-20127 (1998); Hammond, C., et al., J. Immunol. 161:3282-3291 (1998)), on vesicular MIIC-derived exosomes (Raposo, G., et al., J. Exp. Med. 183:1161-1172 (1996)) and on exosomes of dendritic cells (Thery, C. et al., J. Cell Biol. 147:599-610 (1999)). In addition, cell surface expression of CD63 is induced or strongly increased after activation of platelets, neutrophils and basophils, following mobilization of the intracellular pool (Boucheix, C., et al., Cell Mol. Life. Sci., 58:1189-1205 (2001); Lopez, S., et al., Clin. Exp. Immuol. 101(1):25-32 (1995)).

Mast cell FcεRI can be saturated with monoclonal IgE antibodies and can be crosslinked by appropriate antigen, which leads to mast cell activation. Monoclonal antibodies are purified from culture supernatants or mouse ascitic fluid (produced by injection of antibody-producing cells into immunocompromised mice using standard techniques, such as those described in Kohler and Milstein, Nature 256:495-497 (1975); Kozbar et al., Immunology Today 4:72 (1983); and Cole et al., Monoclonal Antibodies and Cancer Therapy, Alan R. Liss, Inc., pp. 77-96 (1985)). Crosslinking by the antigen (protein binding to the IgE) normally induces cell degranulation, which can be quantitated by enzyme assay or radioactivity release assay. As depicted in FIG. 1, anti-CD63 antibodies (e.g., mAb 7A6) inhibited FcεRI-induced degranulation (similar results observed with anti-CD63 mAb 12A2 and mAb 12A10).

In one embodiment, the invention is a method of treating an allergic condition in a mammal comprising administering to the mammal an effective amount of an agent that inhibits a function of CD63.

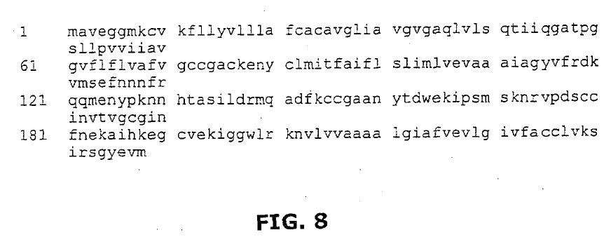

As described herein, CD63 refers to naturally occurring or endogenous CD63 proteins (e.g., mammalian CD63 proteins) and to proteins having an amino acid sequence that is the same as that of a naturally occurring or endogenous corresponding CD63 protein (e.g., recombinant proteins, synthetic proteins (e.g., produced using the methods of synthetic organic chemistry)). Accordingly, as defined herein, the terms include mature CD63 protein, polymorphic or allelic variants, and other isoforms of a mammalian CD63 (e.g., produced by alternative splicing or other cellular processes), and modified or unmodified forms of the foregoing (e.g., glycosylated, unglycosylated, lipidated). Naturally occurring or endogenous mammalian CD63 proteins include wild type proteins such as mature receptor, polymorphic or allelic variants and other isoforms that occur naturally in mammals (e.g., humans, non-human primates). Such proteins, for example, can be recovered or isolated from a source that naturally produces mammalian CD63. These proteins and mammalian receptor proteins having the same amino acid sequence as a naturally occurring or endogenous corresponding mammalian CD63, are referred to by the name of the corresponding mammal. For example, where the corresponding mammal is a human, the protein is designated as a human CD63 protein (e.g., a recombinant human CD63 produced in a suitable host cell). A human CD63 protein sequence is depicted in FIG. 8 (SEQ ID NO:2).

Mast cells are a major cell in allergic reactions and allergic conditions. As described herein, “allergic condition” refers to any condition resulting from antigen activation of mast cells, basophils or other cells relevant for inflammatory conditions (e.g., dendritic cells, macrophages, neutrophils, eosinophils) that results in an “allergic reaction” or state of hypersensitivity and influx of inflammatory and immune cells (e.g., neutrophils, eosinophils, monocytes, lymphocytes). Suitable allergic conditions and inflammatory diseases or disorders that can be treated with the agents of the invention include, e.g.:
systemic allergic reactions, systemic anaphylaxis or hypersensitivity responses, anaphylactic shock (e.g., in response to a specific antigen), drug allergies (e.g., to penicillin, cephalosporins) and insect sting allergies;
respiratory allergic diseases, such as asthma (including allergic asthma, bronchial asthma, exercise-induced asthma (EIA), chemical-induced asthma and status asthmaticus), hypersensitivity lung diseases, hypersensitivity pneumonitis and interstitial lung diseases (ILD) (e.g., idiopathic pulmonary fibrosis, ILD associated with rheumatoid arthritis, or other autoimmune conditions);
rhinitis, hay fever, conjunctivitis, allergic rhinoconjunctivitis and vaginitis;
skin and dermatological disorders, including psoriasis and inflammatory dermatoses, such as dermatitis, eczema, atopic dermatitis, allergic contact dermatitis, dermatitis herpetiformis, linear IgA disease, acute and chronic urticaria and scleroderma;
vasculitis (e.g., necrotizing, cutaneous, and hypersensitivity vasculitis);
spondyloarthropathies; and
intestinal reactions of the gastrointestinal system (e.g., inflammatory bowel diseases such as Crohn's disease, ulcerative colitis, ileitis, enteritis, nontropical sprue and celiac disease).
   Other allergic or inflammatory responses can also be treated with the methods of the invention. For example, mast cell involvement in autoimmune disease (e.g., autoimmune diseases involving auto-antibodies) has recently been reported (Benoist, C., et al., Nature 420(6917):875-878 (2002)). In addition, CD63 has been shown to be upregulated on polymorphonuclear neutrophils in patients with rheumatoid arthritis (Lopez, S., et al., Clin. Exp. Immuol. 101(1):25-32 (1995)) and on platelets (Joseph, J. E., et al., Br. J. Haematol. 115(2):451-459 (2001)). The in vivo data described herein support the use of the agents described herein for treatment of autoimmune disease (e.g., autoimmune diseases involving auto-antibodies, arthritis (e.g., rheumatoid arthritis, psoriatic arthritis), antiphospholipid syndrome and lupus erythematosus). Other allergic or inflammatory responses that can be treated using the methods of the invention include but are not limited to, e.g.:
autoimmune diseases, such as myasthenia gravis, diabetes, including diabetes mellitus and juvenile onset diabetes, glomerulonephritis and other nephritides, (e.g., antibody-mediated nephropathy (e.g., IgA nephropathy (Berger's Disease))), bullous pemphigoid, autoimmune thyroiditis, Behcet's syndrome; autoimmune neurological diseases (e.g., multiple sclerosis), ankylosing spondylitis, sarcoidosis, Sjögren's syndrome, systemic and cutaneous mastocytosis, dermatomyositis, scleroderma, polymyositis, biliary cirrhosis, autoimmune thyroiditis, autoimmune hepatitis; and
neoplastic diseases (e.g. CD63-expressing lymphomas or leukemias).

In one embodiment, the invention is a method of treating an allergic condition that is selected from the group consisting of asthma, rhinitis, hay fever, conjunctivitis, atopic dermatitis, atopic eczema, systemic allergic reactions and intestinal reactions of the gastrointestinal system. In another embodiment, the invention is a method of treating an autoimmune disease (e.g., multiple sclerosis, rheumatoid arthritis, mastocytosis).

Agents described herein can be any molecule that modifies (e.g., inhibits, enhances) a CD63 function. In one embodiment, the invention utilizes an agent that inhibits a CD63 function. Suitable agents for use in the methods of the invention include proteins (e.g., antibodies and antigen-binding fragments thereof), peptides, peptidomimetics, small organic molecules, nucleic acids (e.g., RNAi, antisense oligonucleotides) and ribozymes. In one embodiment, the agent is an antibody or antigen-binding fragment (e.g., an antibody that binds to CD63). The antibody of the invention can be polyclonal or monoclonal, and the term “antibody” is intended to encompass both polyclonal and monoclonal antibodies. The terms polyclonal and monoclonal refer to the degree of homogeneity of an antibody preparation, and are not intended to be limited to particular methods of production. The term “antibody” as used herein also encompasses functional fragments of antibodies, including fragments of chimeric, human, humanized, primatized, veneered or single chain antibodies. Functional fragments (e.g., antigen-binding fragments), including, but not limited to Fv, Fab, Fab′ and F(ab′)₂ fragments are encompassed by the invention. Such fragments can be produced by enzymatic cleavage or by recombinant techniques. For example, papain or pepsin cleavage can generate Fab or F(ab′)₂ fragments, respectively. Other proteases with the requisite substrate specificity can also be used to generate Fab or F(ab′)₂ fragments. Antibodies can also be produced in a variety of truncated forms using antibody genes in which one or more stop codons has been introduced upstream of the natural stop site. For example, a chimeric gene encoding a F(ab′)₂ heavy chain portion can be designed to include DNA sequences encoding the CH₁ domain and hinge region of the heavy chain.

Single chain antibodies, and chimeric, humanized or primatized (CDR-grafted), or veneered antibodies comprising portions derived from different species, and the like, are also encompassed by the present invention and the term “antibody”. The various portions of these antibodies can be joined together chemically by conventional techniques, or can be prepared as a contiguous protein using recombinant DNA technology. For example, nucleic acids encoding a chimeric or humanized chain can be expressed to produce a contiguous protein. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0 451 216 B1; and Padlan, E. A. et al., EP 0 519 596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single chain antibodies.

Humanized antibodies can be produced using synthetic or recombinant DNA technology using standard methods or other suitable techniques. Nucleic acid (e.g., cDNA) sequences coding for humanized variable regions can also be constructed using PCR mutagenesis methods to alter DNA sequences encoding a human or humanized chain, such as a DNA template from a previously humanized variable region (see e.g., Kamman, M., et al., Nucl. Acids Res., 17: 5404 (1989)); Sato, K., et al., Cancer Research, 53: 851-856 (1993); Daugherty, B. L. et al., Nucleic Acids Res., 19(9): 2471-2476 (1991); and Lewis, A. P. and J. S. Crowe, Gene, 101: 297-302 (1991)). Using these or other suitable methods, variants can also be readily produced. In one embodiment, cloned variable regions can be mutated, and sequences encoding variants with the desired specificity can be selected (e.g., from a phage library; see e.g., Krebber et al., U.S. Pat. No. 5,514,548; Hoogenboom et al., WO 93/06213, published Apr. 1, 1993).

Antibodies that are specific for mammalian (e.g., human) CD63 can be raised against an appropriate immunogen. For example, antibodies that specifically bind mammalian CD63, such as isolated and/or recombinant human CD63 or portions thereof (including synthetic molecules, such as synthetic peptides), can be raised. As demonstrated herein, antibodies can also be raised by immunizing a suitable host (e.g., mouse) with cells that express CD63 (e.g., RBL-2H3 cells). In addition, cells expressing a recombinant mammalian CD63, such as transfected cells, can be used as immunogens or in a screen for antibody which binds thereto (see e.g., Chuntharapai et al., J. Immunol., 152: 1783-1789 (1994); Chuntharapai et al., U.S. Pat. No. 5,440,021).

Preparation of Immunizing Antigen, and Polyclonal and Monoclonal Antibody production can be performed using any suitable technique. A variety of methods have been described (see e.g., Kohler et al., Nature, 256: 495-497 (1975) and Eur. J. Immunol. 6: 511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer '94), Ausubel, F. M. et al., Eds., (John Wiley & Sons: New York, N.Y.), Chapter 11, (1991)). Where monoclonal antibodies are desired, a hybridoma is generally produced by fusing a suitable immortal cell line (e.g., a myeloma cell line such as SP2/0, P3X63Ag8.653 or a heteromyeloma) with antibody-producing cells. Antibody-producing cells can be obtained from the peripheral blood or, preferably the spleen or lymph nodes, of humans or other suitable animals immunized with the antigen of interest. The fused cells (hybridomas) can be isolated using selective culture conditions, and cloned by limiting dilution. Cells that produce antibodies with the desired specificity can be selected by a suitable assay (e.g., ELISA).

Other suitable methods of producing or isolating antibodies of the requisite specificity can be used, including, for example, methods which select recombinant antibody from a library (e.g., a phage display library). In one embodiment, the method of the invention utilizes an agent that is a human antibody (e.g., a human anti-CD63 antibody). Methods for generating human antibodies are known in the art. For example, transgenic animals capable of producing a repertoire of human antibodies (e.g., XenoMouse® (Abgenix, Fremont, Calif.), TransChromo Mouse™ (TC Mouse™) or second generation KM Mouse™ (Kirin, Japan)) can be produced using suitable methods (see e.g., Jakobovits et al., Proc. Natl. Acad. Sci. USA, 90: 2551-2555 (1993); Jakobovits et al., Nature, 362: 255-258 (1993); Ishida, I., et al., Cloning Stem Cells 4(1):91-102 (2002)). In the TransChromo™ method, human chromosomes are introduced into mouse cells, which are in turn injected into fertilized mouse eggs and implanted into a mouse uterus. The mouse with human chromosomes is thus able to produce human antibodies. Additional methods that are suitable for production of transgenic animals capable of producing a repertoire of human antibodies have also been described (e.g., Lonberg et al., U.S. Pat. No. 5,545,806; Surani et al., U.S. Pat. No. 5,545,807; Lonberg et al., WO97/13852).

In one embodiment, the invention is a method of treating an allergic condition in a mammal comprising administering to the mammal an effective amount of an agent that inhibits a function of CD63, wherein the agent is an antibody. In another embodiment, the antibody is able to inhibit degranulation (e.g., as measured by release of serotonin (e.g., for rodents), histamine, β-hexosaminidase) at a concentration of 10 μg/ml or less (e.g., using RBL-2H3 cells and measuring serotonin release using the assay described herein, using RBL-2H3 cells and measuring histamine release using the assay described by Kitani et al. (Kitani et al., J. Biol. Chem. 266(3): 1903-1909 (1991)). In other embodiments, the antibody is able to significantly inhibit degranulation (e.g., using RBL-2H3 cells and measuring serotonin or histamine release) using concentrations of 8 μg/ml or less, 5 μg/ml or less, 4 μg/ml or less, 2 μg/ml or less, or 1 μg/ml or less.

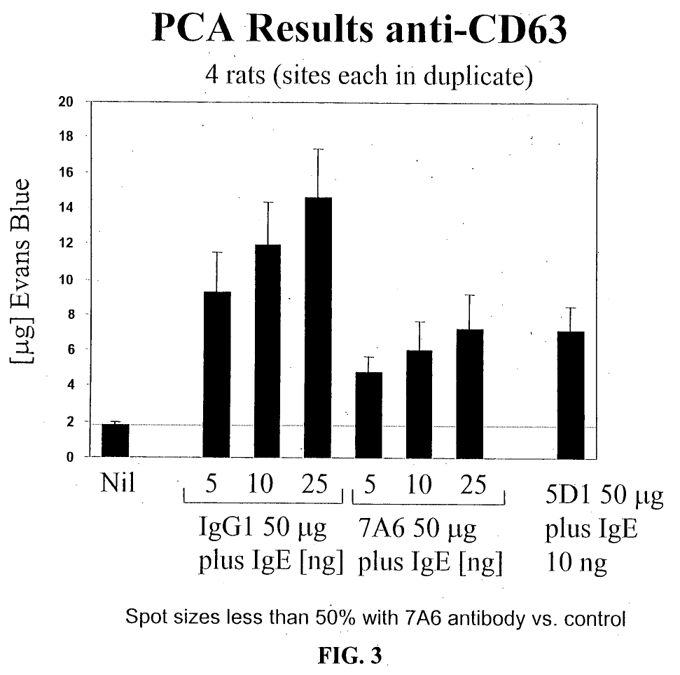

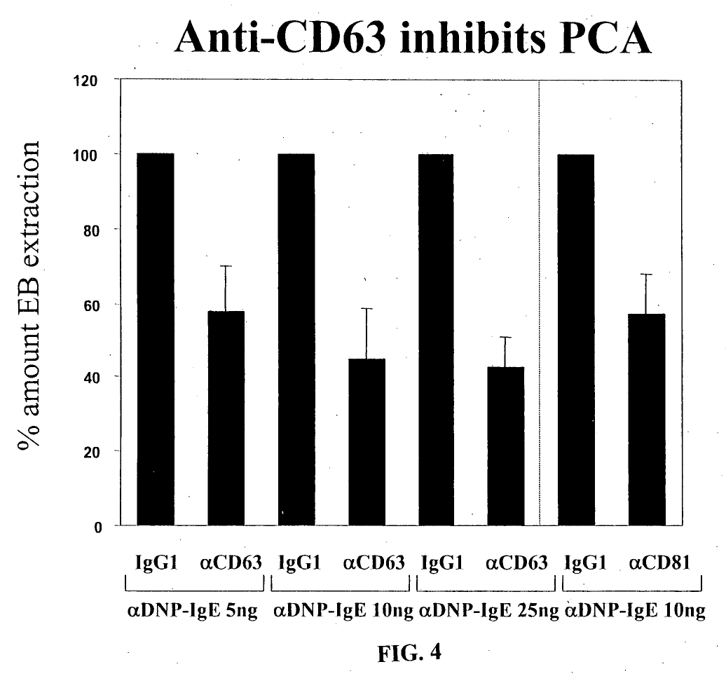

In another embodiment, the invention is a method of inhibiting passive cutaneous anaphylaxis in a mammal comprising administering to the mammal an effective amount of an agent that inhibits a function of CD63. As demonstrated herein, administration of anti-CD63 antibodies resulted in a greater than 50% inhibition of the anaphylactic reaction (FIGS. 3 and 4). In still another embodiment, the invention is a method of inhibiting anaphylaxis in a mammal (e.g., human) comprising administering to the mammal an effective amount of an agent that inhibits a function of CD63.

The present invention can be used to develop and/or screen for agents (e.g., antibodies, RNAi, antisense oligonucleotides, ribozymes, small organic molecules, peptides, peptidomimetics) that inhibit the allergic process, and/or develop compounds for the treatment of allergies, anaphylactic reactions and related diseases. In one embodiment, the invention is an assay for identifying an agent that alters the interaction of CD63 and a binding partner of CD63 (e.g., integrin (e.g., β1-integrin), ProHB-EGF, CD9P-1, tetraspanin (e.g., CD81, CD9)). In the assay, a cell bearing CD63 (e.g., cell-surface CD63), a binding partner of CD63 and an agent to be tested, are combined under conditions suitable for binding of CD63 to the binding partner and the level of CD63-mediated signal transduction is determined, wherein if the level of CD63-mediated signal transduction is altered relative to a control, the agent alters CD63-mediated signal transduction.

Thus, agents can be screened and/or tested for their capacity to modulate (e.g., inhibit, enhance) a function of CD63. Such agents can be individually screened or one or more agents can be tested simultaneously using suitable binding and/or functional assays. Where a mixture of compounds is tested, the compounds selected by the processes described can be separated (as appropriate) and identified using suitable methods (e.g., sequencing, chromatography). The presence of one or more compounds (e.g., inhibitor, promoter) in a test sample can also be determined according to these methods.

Inhibitors or promoters of a function of CD63, e.g., those identified by methods described herein, can be assessed to determine their effect on cell surface receptor signaling. For example, a cell bearing CD63 and an appropriate cell surface receptor (e.g., FcεRI or FcγRIII) are combined with an inhibitor or promoter of a CD63 function under conditions suitable for signal transduction of the cell surface receptor. The level or extent of cell surface receptor signaling can be measured using standard methods and compared with the level or extent of cell surface receptor signaling in the absence of the inhibitor or promoter (control). An increase in the level or extent of cell surface receptor signaling relative to the control indicates that the agent is a promoter of cell surface receptor signaling (e.g., an enhancer of CD63 function); a decrease in the level or extent of cell surface receptor signaling relative to the control indicates that the agent is an inhibitor of cell surface receptor signaling (e.g., an inhibitor of CD63 function). A suitable control, includes, e.g., the absence of the agent.

Cell surface receptor signaling can be measured directly, such as by measuring the level or amount of an associated signaling molecule, or indirectly, such as by a functional assay measuring level or amount of degranulation or passive cutaneous anaphylaxis.

Suitable cells and cell lines that can be used in the assays and methods described herein include, e.g., any cells or cell lines that express CD63 and an appropriate antigen receptor (e.g., Fc antigen receptor). In one embodiment, the assay is performed on cells or cell lines selected from the group of consisting of mast cells, neutrophils, basophils, granulocytes, dendritic cells, eosinophils, mast cell-derived cell lines, neutrophil-derived cell lines, basophilic leukemia cell-derived cell lines, granulocyte-derived cell lines, dendritic cell-derived cell lines and eosinophil-derived cell lines. Other suitable cells and/or endogenous sources of CD63 include, e.g., isolated cells that express CD63 (e.g., platelet lysosomes (Azorsa, D., et al., Blood 78:280-284 (1991)), Weibel-Palade bodies of endothelial cells (Vischer, U. M., et al., Blood 82:1184-1191 (1993)), azurophil granules of neutrophil granulocytes (Fitter, S., et al., Biochem J. 338:61-70 (1999)), major histocompatibility (MHC) class II-enriched compartments (MIIC) of B lymphocytes (Escola, J. M., et al., J. Biol. Chem. 273:20121-20127 (1998); Hammond, C., et al., J. Immunol. 161:3282-3291 (1998)), vesicular MIIC-derived exosomes (Raposo, G., et al., J. Exp. Med. 183:1161-1172 (1996)) and exosomes of dendritic cells (Thery, C. et al., J. Cell Biol. 147:599-610 (1999)).

Appropriate functional assays for use in determining the level of Fc antigen receptor-mediated signal transduction include, e.g., assays that measure release of proinflammatory mediators (e.g., cytokines (e.g., IL-1, IL-2, IL-3, IL-4, IL-5, IL-6, IL-10, IL-13, IL-14, GM-CSF, TGF-β, TNF-αc, NGF, MIP-1β, MCP-1)), leukotriene synthesis, cytokine synthesis, degranulation (e.g., as measured by release of serotonin (e.g., for rodents), histamine, β-hexosaminidase), calcium mobilization, protein tyrosine phosphorylation, enzymatic activity of protein kinases and antigen-presenting function of dendritic cells (e.g., using mixed leukocyte reaction (MLR)). These functional assays can be used to evaluate an agent's ability to inhibit Fc antigen receptor signaling. Agents that are identified may be tested for in vivo efficacy, e.g., using an appropriate model (e.g., the PCA model described herein)).

As described herein, “a function of CD63” includes any function of CD63 that results in the production of an allergic, hypersensitivity or inflammatory reaction. Such CD63 functions include, e.g., binding of CD63 to other molecules (e.g., integrins (e.g., P1 integrins), ProHB-EGF, CD9P-1, tetraspanins (e.g., CD81, CD9)), adhesion, signaling activities, induction of cellular responses (e.g., inflammatory mediator release), migration, antigen presentation (e.g., on dendritic cells). In one embodiment the CD63 function is binding to an integrin (e.g., a β1-integrin). An agent can be studied in one or more suitable functional and/or binding assays to determine if said agent can modulate (e.g., inhibit (reduce or prevent) or promote) one or more binding, signaling activities or cellular responses induced upon activation or binding of CD63. Thus, for example and as described herein, it is possible to assay the effect of an agent on adhesion of a cell that expresses CD63 (e.g., RBL-2H3) to fibronectin (a ligand for integrins). It is also possible to assay one or more agents for modulation of direct binding of CD63 to molecules with which it is known to interact (e.g., integrins (e.g., α3β1, α6β1, Integrin α_{L}β₂), ProHB-EGF, CD9P-1, tetraspanins (e.g., CD81, CD9)) (Boucheix, C., et al., Cell Mol. Life. Sci. 58:1189-1205 (2001)).

Agents that modulate binding of CD63 and/or that modulate the function of CD63 (e.g., signaling, adhesion, activation) can be used in the therapeutic methods described herein and can be identified, for example, by screening libraries or collections of molecules, such as, the Chemical Repository of the National Cancer Institute, in assays described herein or using other suitable methods. Combinatorial libraries of compounds (e.g., organic compounds, recombinant or synthetic peptides, “peptoids”, nucleic acids) produced by combinatorial chemical synthesis or other methods can be tested (see e.g., Zuckerman, R. N. et al., J. Med. Chem., 37: 2678-2685 (1994) and references cited therein; see also, Ohlmeyer, M. H. J. et al., Proc. Natl. Acad. Sci. USA 90:10922-10926 (1993) and DeWitt, S. H. et al., Proc. Natl. Acad. Sci. USA 90:6909-6913 (1993), relating to tagged compounds; Rutter, W. J. et al. U.S. Pat. No. 5,010,175; Huebner, V. D. et al., U.S. Pat. No. 5,182,366; and Geysen, H. M., U.S. Pat. No. 4,833,092). Where compounds selected from a combinatorial library by the present method carry unique tags, identification of individual compounds by chromatographic methods is possible.

In addition to screening for agents, agents can be designed and generated using available technology. For example, suitable agents that eliminate or reduce expression of CD63 can be produced using, e.g., RNA interference technology (also known as RNAi, post-transcriptional gene silencing and quelling). RNA interference, commonly referred to as RNAi, is a phenomenon resulting from the overexpression or misexpression of transgenes, or from the deliberate introduction of double-stranded RNA into cells (see, e.g., Fire, A, Trends Genet. 15:358-363 (1999); Sharp, P. A., Genes Dev. 13:139-141 (1999); Hunter, C., Curr. Biol. 9:R440-R442 (1999); Baulcombe, D. C., Curr. Biol. 9: R599-R601 (1999); Vaucheret et al., Plant J. 16:651-659 (1998); Scherr, M., et al., Curr. Med. Chem. 10(3):245-256 (2003)). RNAi provides a way of specifically and potently inactivating a gene (e.g., CD63), and is proving a powerful tool for investigating gene function. Use of RNAi for reducing gene expression and suitable vectors for producing RNAi are known in the art (e.g., U.S. Patent Publication No. 200030084471 and Scherr, M., et al., Curr. Med. Chem. 10(3):245-256 (2003)).

In other embodiments, gene expression can be reduced through the use of antisense technology (e.g., antisense oligonucleotides) and/or ribozymes. Use of antisense technology and ribozymes is well known in the art and is described, e.g., by Kurreck, J., Eur. J. Biochem. 270(8):1628-1644 (2003); Folini, M, Curr. Med. Chem. Anti-Canc. Agents 2(5):605-612 (2002); the teachings of both of which are incorporated herein by reference in their entirety).

In one embodiment, the invention is an agent, wherein the agent is an inhibitor (e.g., an RNAi molecule, an antisense oligonucleotide, a ribozyme) of a function of CD63.

As described herein, anti-CD63 antibodies inhibited FcεRI-mediated degranulation. However, as with antibodies to the related tetraspanin CD81, anti-CD63 antibodies did not affect tyrosine phosphorylation or calcium mobilization events (see U.S. Pat. No. 6,423,501).

Thus, CD63 and CD81 are tetraspanins that have a role in the progression of allergic reaction. Tetraspanins (e.g., CD81, CD63) are known to bind integrins, which are important receptor proteins as they facilitate and promote cell binding and responses to the extracellular matrix. Functional integrins consist of two transmembrane glycoprotein subunits that are non-covalently bound. Those subunits are called alpha and beta. The alpha subunits all have some homology to each other, as do the beta subunits. The receptors always contain one alpha chain and one beta chain and are thus called heterodimeric. A large number of alpha and beta subunits have been identified, which combine in different ways to form at least 22 natural integrins. Integrins can adhere to (bind) an array of ligands (e.g., fibronectin and laminin) and are involved in a wide range of biological functions (e.g., maintenance of tissue homeostasis through binding to matrix proteins, leukocyte migration, cell activation).

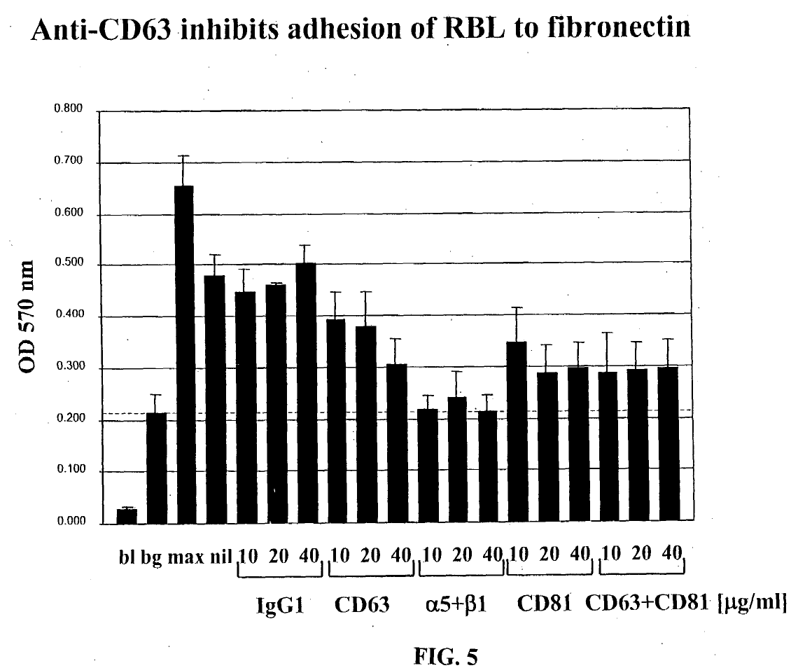

As demonstrated herein, anti-CD63 antibodies were able to significantly reduce the number of RBL-2H3 cells that adhered to extracellular matrix (ECM) proteins (e.g., fibronectin) when tested in an adhesion assay (FIG. 5). Thus, inhibition of the interaction of tetraspanins (e.g., CD63, CD81) with integrins (for example, using the agents of the invention) represents a desirable therapeutic target for inhibiting downstream events that lead to allergic reactions. Thus, in one embodiment, the invention is a method of treating an allergic condition in a mammal comprising administering to the mammal an effective amount of an agent that inhibits the interaction of a tetraspanin (e.g., CD63, CD81) with an integrin (e.g., a β1 integrin, β2 integrin) or other binding partner of the tetraspanin (e.g., ProHB-EGF, CD9P-1, another tetraspanin (e.g., CD81, CD9)). Tetraspanins that are expressed on granulocytes, mast cells or basophils are likely candidates for targeting, as are tetraspanins that are known to interact with CD63 and/or CD81 (e.g., CD9). Using the methods described herein and routine experimentation, the person of ordinary skill in the art could screen and/or design agents that inhibit the interaction of a tetraspanin (e.g., CD63, CD81, CD9) and an integrin (e.g., a 1 integrin) to isolate other therapeutic agents for inhibiting allergic reactions.

In another embodiment, the present invention is a method of inhibiting or enhancing cell surface receptor signaling (e.g., FcεRI-mediated, FcγRIII-mediated signaling) comprising administering an agent that modulates (e.g., inhibits, enhances) a function of a tetraspanin (e.g., CD63, CD81). The method of modulating cell surface receptor signaling comprises contacting a cell with an effective amount of an agent that modulates (e.g., inhibits, enhances) a function of the tetraspanin. Appropriate cells are any cell type that expresses, or has been designed to express (e.g., by transfection or genetic engineering), the tetraspanin and a suitable cell surface receptor (e.g., FcεRI, FcγRIII).

As described, inhibition of a tetraspanin (e.g., CD63, CD81) function (e.g., binding (e.g., to integrins or other binding partners), adhesion, signaling, induction of cellular responses) that inhibits mast cell degranulation is useful in methods of treating allergic conditions or inflammatory disorders. In contrast, enhancement of a tetraspanin (e.g., CD63, CD81) function, which induces mast cell degranulation, is useful in inducing an inflammatory response, for example, in response to bacterial or parasite infection. It may be clinically beneficial to enhance cell surface receptor signaling in a mammal by enhancing a function of a tetraspanin (e.g., CD63, CD81, CD9), in conditions where an inflammatory response and/or release of leukotrienes and cytokines is beneficial (e.g., in host defense against parasites and bacteria). Agents that enhance a function of a tetraspanin can be isolated, e.g., using the assays and methods described herein.

As used herein, “inhibit” is intended to encompass any qualitative or quantitative reduction in a measured effect or characteristic, including complete prevention, relative to a control. As used herein, “enhance” is intended to encompass any qualitative or quantitative increase in a measured effect or characteristic relative to a control. An “effective amount” of a given agent is intended to mean an amount sufficient to achieve the desired effect, e.g., the desired therapeutic effect, under the conditions of administration, such as an amount sufficient for inhibition or enhancement of a CD63 function.

The present invention also relates to preparations for use in the methods of the invention (e.g., the treatment of allergic diseases and inflammatory disorders). Such preparations may include, e.g., an inhibitor or enhancer of a tetraspanin (e.g., CD63) function, together with a physiologically-acceptable carrier. Such preparations may also optionally contain physiologically-acceptable adjuvants.

According to the methods, a therapeutically effective amount of an agent (e.g., an anti-CD63 antibody) can be administered to an individual using an appropriate route of administration, either alone or in combination with another drug. A variety of routes of administration are possible including, but not limited to, oral, dietary, topical, parenteral (e.g., intravenous, intraarterial, intramuscular, subcutaneous injection), and inhalation (e.g., intrabronchial, intranasal or oral inhalation, intranasal drops) routes of administration. Determination of an appropriate route of administration depends on the particular agent to be administered and the disease or condition to be treated and is within the skill of the ordinary person in the art. For respiratory allergic diseases (e.g., asthma), inhalation is a preferred mode of administration as it targets the agent to the pulmonary system.

Formulation of an agent to be administered will vary according to the route of administration selected (e.g., solution, emulsion, capsule). An appropriate composition comprising the agent to be administered can be prepared in a physiologically-acceptable vehicle or carrier. For solutions or emulsions, suitable carriers include, for example, aqueous or alcoholic/aqueous solutions, emulsions or suspensions, including saline and buffered media. Parenteral vehicles can include, e.g., sodium chloride solution, Ringer's dextrose, dextrose and sodium chloride, lactated Ringer's or fixed oils. Intravenous vehicles can include various additives, preservatives, or fluid, nutrient or electrolyte replenishers, and the like (See generally, Remington's Pharmaceutical Sciences, 17th Edition, Mack Publishing Co., PA, 1985). For inhalation, the agent can be solubilized and loaded into a suitable dispenser for administration (e.g., an atomizer, nebulizer or pressurized aerosol dispenser).

Furthermore, where the agent is a protein or peptide, the agent can be administered via in vivo expression of the recombinant protein. In vivo expression can be accomplished via somatic cell expression according to suitable methods (see, e.g., U.S. Pat. No. 5,399,346). In this embodiment, a nucleic acid encoding the protein can be incorporated into a retroviral, adenoviral or other suitable vector (preferably, a replication-deficient infectious vector) for delivery, or can be introduced into a transfected or transformed host cell capable of expressing the protein for delivery. In the latter embodiment, the cells can be implanted (alone or in a barrier device), injected or otherwise introduced in an amount effective to express the protein in a therapeutically effective amount.

The methods of the invention have advantages over current treatments of allergic conditions (e.g., using antihistamines, corticosteroids). Unlike antihistamines (which are also only designed to treat certain aspects of an allergic reaction (e.g., histamine release)), the agents of the present invention (e.g., anti-CD63 antibodies) target a cell surface molecule that is expressed on the surface of cells that are important in generating allergic reactions (e.g., activated mast cells, basophils, eosinophils, macrophages). Moreover, given that, in vivo, CD63 is only surface-expressed on a limited subset of cells (e.g., activated mast cells, basophils, eosinophils, macrophages), targeting of CD63 (e.g., using an anti-CD63 antibody) may have reduced adverse side effects (both in number and severity) as compared to current drugs (e.g., antihistamines) that merely target effector molecules (e.g., antihistamines). Also, an antibody drug (e.g., an anti-CD63 human antibody) may also have a longer in vivo half life than current therapeutic agents for treating allergy and therefore may need to be applied less often.

The following Examples are offered for the purpose of illustrating the present invention and are not to be construed to limit the scope of this invention. The teachings of all references cited herein, which have not already been incorporated by reference in their entirety, are hereby incorporated by reference in their entirety.

### EXAMPLES

### Immunizations, Fusions, and FACS

Female BALB/c mice (4-8 weeks old) were immunized intraperitoneally with 25×10⁶ RBL-2H3 cells (which most closely resemble immature rat mucosal mast cells) emulsified in complete Freund's adjuvant or 50×10⁶ RBL-2H3 cells in phosphate buffered saline (PBS). Mice were boosted intraperitoneally after 2 weeks with 40×10⁶ RBL-2H3 cells emulsified in incomplete Freund's adjuvant or in PBS. For the final immunizations, animals were injected with 20−40×10⁶ RBL-2H3 cells intraperitoneally at day −4 (fusion=day 0) and intravenously at day −3. Spleen cell preparations were fused with either NS-1 or SP2/0 myeloma cells in polyethylene glycol and plated onto normal BALB/c spleen feeder cells. To enhance the development of the hybridomas, S. typhimurium mitogen (Ribi ImmunoChem Research, Inc., Hamilton, Mont.) was included in the culture medium from days 0-10. Hybridoma supernatants were tested after day 14 by flow cytometry for binding to RBL-2H3 cells using FITC-conjugated goat anti-mouse F(ab′)₂-specific antibody (Jackson ImmunoResearch Laboratories, Inc., West Grove, Pa.) and analyzed by flow cytometry on a FACSCAN™ flow cytometer (Becton-Dickinson, San Jose, Calif.).

A panel of monoclonal antibodies was produced and the hybridoma supernatants were screened for their ability to inhibit serotonin release upon high affinity IgE receptor (FcεRI) triggering on RBL-2H3 (a standard assay for mast cell degranulation; see below). Hybridoma clones that produced antibodies that were able to inhibit serotonin release were further expanded and injected into the peritoneal cavity of mice in order to produce ascites containing the antibodies. Larger amounts of antibodies were purified from ascites using immunoaffinity chromatography and their isotypes were determined (e.g., IgG1 for 7A6 and 12A10).

### Immunoprecipitation

Immunoprecipitation of CD63 proteins using mAb AD1 (Kitani, S., et al., J. Biol. Chem. 266(3):1903-1909 (1991)), mAb 7A6, mAb 12A2 and mAb 12A10 was performed as follows. RBL-2H3 cells were trypsinized, washed once with complete culture medium, washed twice with PBS and then lysed at 40×10⁶ cells/ml on ice using 50 mM Tris, pH 7.4, +130 mM NaCl+5 mM EDTA+1% Triton X-100 containing 10 μg/ml aprotinin, leupeptin and pepstatin A. Postnuclear lysates were prepared by centrifugation at 14,000 g at 4° C. and preclearing was performed using protein G Sepharose (Amersham Bioscience, Piscataway, N.J.) for 1 hour. The lysates were then incubated with specific antibodies (mAb AD1, mAb 7A6, mAb 12A2 or mAb 12A 10) or isotype control antibodies at 10-20 μg/ml overnight. Antibodies were captured using protein G Sepharose for 1 hour. The beads were then washed and the bound proteins eluted using LDS sample buffer (Invitrogen, Carlsbad, Calif.) and subsequent boiling.

### Western Blots

Whole cell lysates or immunoprecipitated proteins were loaded on 4-12% or 10% NuPage Bis-Tris gels (Invitrogen, Carlsbad, Calif.), transferred to a PVDF membrane, blocked with either 5% milk or 3% BSA (for antiphosphotyrosine blots) and immunoblotted with 1 μg/ml of anti CD63 mAb (e.g., mAb AD1, mAb 7A6, mAb 12A2 or mAb 12A10) or 4G10 mAb (for antiphosphotyrosine blots). After incubation with a goat anti-mouse secondary antibody, membranes were developed using ECL substrate (Amersham Bioscience, Piscataway, N.J.).

### Deglycosylation Experiments

CD63 was immunoprecipitated using mAb 7A6 or mAb 12A 10 and eluted using nonreducing LDS sample buffer (Invitrogen, Carlsbad, Calif.) and subsequent boiling. The resulting eluted fraction was incubated with 10% of 10×G7 buffer, 1% Triton X-100 and PNGaseF (New England Biolabs, Beverly, Mass.) or O-glycosidase (Roche, Basel, Switzerland) or O-glycosidase plus neuraminidase (Roche, Basel, Switzerland) or N-glycanase for 1-4 hours at 37° C., according to the manufacturers instructions. Deglycosylation with O-glycosidase and neuraminidase was also performed in 20 mM NaPO₄, pH 7.2.

### Cloning and Expression of Rat CD63

Total RNA was isolated from RBL-2H3 cells using RNAzol B (Tel-Test Inc., Friendswood, Tex.) and cDNA was obtained from 1 μg of RNA using Omniscript reverse transcriptase (Qiagen, Valencia, Calif.). N-terminal FLAG-tagged rat CD63, flanked by EcoRI and HindIII sites, was amplified from cDNA using Advantage cDNA polymerase (BD Clontech, Palo Alto, Calif.) and the following primer set:

forward oligonucleotide:

(SEQ ID NO:3)

5′ CAG AAT TCC CAC CAT GGG CGA CTA CAA GGA CGA CGA
TGA CAA GGC GGT GGA AGG AGG AAT GAA GTG TG- 3′;
and
reverse oligonucleotide:

(SEQ ID NO:4)

5′- CAC AAG CTT GGG CTA CAT TAC TTC GTA GCC ACT
CC -3′.

The 762 bp PCR product was subsequently cloned into pCR2.1-TOPO (Invitrogen, Carlsbad, Calif.). After verification by restriction enzyme digest and sequencing, the insert was isolated using EcoRI and HindIII and subcloned in the pBJ1 neo expression vector. 10 μg of linearized and ethanol-purified DNA was used for electroporation of U937 cells (950 μF; 300 V) and selection was initiated 48 hours later with 0.6 μg/ml of G418 (Invitrogen, Carlsbad, Calif.). After establishment of resistant bulk colonies, reactivity of anti-CD63 antibodies (e.g., mAb 7A6, mAb 12A10) with rat CD63-transfected U937 cells and wildtype U937 cells was determined by indirect immunofluorescence with a goat anti-mouse FITC secondary antibody (Jackson Immunoresearch, West Grove, Pa.) and analysis on a FACScalibur flow cytometer (BD Immunocytometry Systems, San Jose, Calif.).

### Serotonin Release Assays

RBL-2H3 cells were loaded with [³H]5-hydroxytryptamine ([³H]serotonin; 0.5 μCi/10⁵ cells) and saturated with DNP-specific IgE (0.2 μg/ml; Sigma) for 16 hours in 24-well tissue culture plates (2×10⁵ cells/well, 37° C., 5% CO₂). At the end of the loading period, cells were incubated with 1-8 μg/ml of control antibody (MOPC 31 c, mouse IgG1 (Sigma, St. Louis, Mo.) or mAb 12A10 or mAb 7A6 (mouse IgG1; anti-rat CD63) for 30 minutes at 37° C. Then, monolayers were washed three times with Modified Tyrode's buffer (135 mM NaCl, 5 mM KCl, 1 mM MgCl₂, 1.8 mM CaCl₂, 0.1% BSA, 5.6 mM glucose, 10 mM Hepes, pH 7.4). In one set of experiments, FcεRI was triggered by adding 50 ng/ml DNP-HSA in Modified Tyrode's buffer and incubating the plates for 20 min at 37° C. with control samples present on each plate. In another set of experiments, FcεRI was triggered by adding 20 nM of phorbol myristate acetate (PMA)+2 μM of ionomycin and incubating for 15 minutes at 37° C. Degranulation was stopped by placing the plates on ice and by the addition of 800 μl of cold Modified Tyrode's buffer per well. 500 μl aliquots were taken from replicate well for scintillation counting. Total cellular incorporation was determined from 0.5% Triton X-100 lysates.

### Adhesion Assays to Extracellular Matrix (ECM) Proteins

96-well tissue culture microplates were coated with 10 μg/ml fibronectin or 20 μg/ml laminin-1 (Sigma, St. Louis, Mo.) in Dulbecco's Phosphate Buffer Saline (PBS) for 16 hours at room temperature, washed once with Dulbecco's PBS and then blocked with 2% BSA in Ca²⁺/Mg²⁺-free Dulbecco's PBS (preheated at 85° C., then cooled down to room temperature) for 2 hours at 37° C. RBL-2H3 cells were starved in culture medium containing no FCS for 16 hours. The cells were subsequently detached with trypsin, washed three times with culture medium containing 0.1% BSA and no FCS, and resuspended at 1×10⁶ cells/ml in this medium. Control antibodies (IgG1) or inhibitory antibodies against tetraspanins (e.g., anti-CD63 mAb 12A10, anti-CD81 mAb 5D1) or β1-integrins (α5+β1 (BD Pharmingen, San Diego, Calif.)) were added at 10-40 μg/ml for 1 hour at 37° C. After washing the 96-well tissue culture microplates two times with Dulbecco's PBS, 1×10⁵ cells in 100 μl of Dulbecco's PBS containing the antibodies mentioned above were added to the wells for 30 minutes (for the fibronectin-coated plates) or 45 minutes (for the laminin-1-coated plates) at 37° C. Cells were then washed carefully once with Dulbecco's PBS and subsequently fixed with 2.5% formaldehyde. After 2 more washes with Dulbecco's PBS, staining was performed for 1 hour at room temperature with 0.1% crystal violet. Excess dye was washed off by washing 5 times with Dulbecco's PBS and cells were lysed in 10% acetic acid for 10 minutes on a shaker at room temperature. Adhesion was then quantified by reading absorption of the incorporated dye at 570 nm.

### Passive Cutaneous Anaphylaxis in Rats

Female CD rats were used in these experiments. Rats were first anesthetized, their back skin hair shaved, and then injected intradermally with 50 μl containing 5-25 ng anti-DNP IgE mixed with 50 μg of control antibody (MOPC 31 c, mouse IgG1 (Sigma, St. Louis, Mo.), mAb 12A10 (mouse IgG1; anti-rat CD63) or mAb 5D1 (mouse IgG1; anti-CD81 (see U.S. Pat. No. 6,423,501)). Control sites received buffer alone (sterile PBS containing 10 μg/ml of mouse serum albumin (Sigma, St. Louis, Mo.)). Sites were marked on the skin for orientation using a red permanent marker. Duplicate test sites were used for each condition. 24 hours after IgE injections, the rats were again anesthetized and injected into one lateral tail vein with 1 ml of sterile 0.9% NaCl solution containing 1 mg/ml DNP-HSA and 1% Evan's Blue dye. 30 minutes after intravenous injection, rats were sacrificed and punch biopsies (0.9 cm diameter) were obtained, minced and extracted three times with hot formamide (80° C., 3 hours). Pooled samples from tissue sites were centrifuged and absorbance at 610 nm (A₆₁₀) was measured. A₆₁₀ values were converted to micrograms of Evan's Blue dye based on a standard curve of dilutions of Evan's Blue in formamide.

### Results

### Identification of the CD63 Molecule as the Antibody Target

Using cross competition of antibody experiments, immunoprecipitation and Western Blot Analysis, it was determined that each of the monoclonal antibodies, mAb 7A6, mAb 12A2 and mAb 12A10, react with a 50-60 kD protein and do not crossreact with FcεRI. This 50-60 kD protein could be deglycosylated using N-glycanase, but not O-glycanase, and deglycosylation revealed a protein of approximately 25 kD.

To test whether this target protein was CD63, two strategies were utilized. The first strategy utilized the monoclonal antibody, mAb AD1, which is known to have binding specificity for rat CD63 (Nishikata, H, et al., J. Immunol. 149:862-870 (1992)). In one set of experiments, mAb 7A6 and mAb 12A10 were used to immunoprecipitate RBL-2H3 cell lysates. The resulting immuoprecipitate was subjected to Western Blot analysis using the monoclonal antibody AD1. For each of the mAb 7A6 and mAb 12A10 immunoprecipitations, CD63 was immunoprecipitated and recognized by AD1. In a second set of experiments, AD1 was used to immunoprecipitate RBL-2H3 cell lysates and the resulting immunoprecipitate was subjected to Western Blot analysis using each of mAb 7A6, mAb 12A2 and mAb 12A10. In all experiments, CD63 was immunoprecipitated by AD1 and recognized by the corresponding anti-CD63 antibody (i.e., mAb 7A6, mAb 12A2 or mAb 12A10).

The second strategy involved cloning rat CD63 from cDNA isolated from RBL-2H3 cells using RT-PCR and expressing rat CD63 in human U937 cells using a eukaryotic expression vector. Reactivity of anti-CD63 antibodies (e.g., mAb 7A6, mAb 12A2, mAb 12A10) with wildtype U937 cells and rat CD63-transfected U937 cells was then analyzed using indirect immunofluorescence. CD63-transfected U937 cells, but not wildtype U937 cells, were recognized by mAb 7A6, mAb 12A2 and mAb 12A10.

Anti-CD63 Suppresses FcεR1-Induced Degranulation But does not Affect PMA/Ionomycin-Induced Degranulation

FcεRI binding of IgG is detectable only when IgG is present in the form of IgG-containing immune complexes that crosslink FcεRI receptors and initiate intracellular signals. As depicted in FIG. 1, DNP-HSA triggers FcεR1-induced degranulation, which is inhibited by anti-CD63 mAb 7A6. However, as depicted in FIG. 2, anti-CD63 mAb 7A6 did not inhibit PMA/ionomycin-induced degranulation. Thus, as with anti-CD81 antibodies (see U.S. Pat. No. 6,423,501), anti-CD63 inhibition is specific for FcεR1 signaling, as degranulation induced by PMA and calcium ionophore ionomycin was unaffected.

### Identification of a Possible Inhibitory Mechanism of Anti-CD63 Antibodies

Proximal FCER1 signaling pathways, assessed by the analysis of tyrosine phosphorylation and calcium mobilization (Turner, H, and Kinet, J. P., Nature 402 (6760 Suppl):B24-B30 (1999)), remain unaffected by preincubation with anti-CD63 antibodies. This also includes the phosphorylation of adaptor proteins known to be involved in degranulation, such as Gab2 and FYB/SLAP-130, which remains intact.

One possibility is that anti-CD63 affects the functions of β1-integrins, which are known to be partner molecules of CD63 (Boucheix, C., et al., Cell Mol. Life. Sci. 58:1189-1205 (2001)). Tetraspanins (e.g., CD63, CD81) are believed to act as organizers of multimolecular complexes by lateral interaction (within the cell membrane) with themselves and other molecules such as β1-integrins (Boucheix, C., et al., Cell Mol. Life. Sci. 58:1189-1205 (2001)). It is believed that these interactions support β1-integrin function (e.g., adhesion, migration). Moreover, adhesion to extracellular matrix (ECM) proteins, or, on a molecular level, the signals resulting from ECM-β1-integrin interactions, are known to be an important supporting factor for mast cell degranulation. Thus, anti-CD63 antibodies may interfere with CD63-β1-integrin interactions, thereby affecting integrin functions and outside-in signaling.

In support of this, it was observed that anti-CD63 antibodies (e.g., mAb 7A6 (FIG. 1), mAb 12A2 and mAb 12A10 (data not shown)) specifically inhibit FcεRI-induced mediator release of adherent RBL-2H3 cells (FIG. 1), but not RBL-2H3 cells in suspension (and also not mediator release induced by PMA and ionomycin (FIG. 2)). Anti-CD63 preincubation did not inhibit FcεRI-induced proximal signals, such as tyrosine phosphorylation and calcium mobilization. However, adhesion of RBL-2H3 cells to ECM proteins, such as fibronectin, has been shown to enhance FcεRI-induced degranulation (Hamawy, M. M., et al., J. Immunol. 149(2):615-621 (1992)) and fibronectin-binding integrins (e.g., a4β1, β5βB) have been shown to be CD63 interaction partners (Boucheix, C., et al., Cell Mol. Life. Sci. 58:1189-1205 (2001)). Thus, anti-CD63 antibodies may affect a “costimulatory” signal derived from adhesion of RBL-2H3 cells to ECM proteins. Additionally, in adhesion assays that measured attachment of RBL-2H3 cells to ECM proteins, such as fibronectin, the addition of anti-CD63 antibodies (e.g., mAb 12A10) and anti-CD81 antibodies (e.g., mAb 5D1) were able to significantly reduce the number of adherent cells (FIG. 5). This lack of a “costimulatory signal” may explain the reduced ability of the cells to degranulate.

### In Vivo Effectiveness of Anti-CD63 Antibodies in Suppressing Allergic Reactions

The in vivo effects of anti-CD63 antibodies were tested using a rat passive cutaneous anaphylaxis (PCA) model, a classical system for studying mast cell activation in vivo (Dombrowicz, D., et al., J. Clin. Invest. 99:915-925 (1997); Wershil, B. K., et al., J. Immunol. 154:1391-1398 (1995); Fleming, T. J., et al., J. Exp. Med. 186(8):1307-1314 (1997)). In this model, an area of skin on the back of a rat is shaved and antigen-specific IgE is injected into this area. After 24 hours, an immediate allergic reaction is elicited by intravenous injection of antigen plus a dye. The local sites of anaphylactic reaction are identified by extravasation of the dye and the reaction can be quantified by extraction of the dye. As depicted in FIGS. 3 and 4, coinjection of anti-CD63 antibodies with various doses of antigen-specific IgE resulted in a greater than 50% inhibition of the anaphylactic reaction. These results were comparable to the inhibition observed with anti-CD81 mAb 5D1 (FIGS. 3 and 4; see also U.S. Pat. No. 6,423,501).

While this invention has been particularly shown and described with references to preferred embodiments thereof, it will be understood by those skilled in the art that various changes in form and details may be made therein without departing from the scope of the invention encompassed by the appended claims.

What is claimed is:

1. A human antibody that binds to CD63.

2. The antibody of claim 1 wherein the antibody is a monoclonal antibody.

3. An agent that inhibits a function of CD63.

4. The agent of claim 3 wherein the agent is selected from the group consisting of an RNAi molecule, an antisense oligonucleotide and a ribozyme.

5. The agent of claim 3 wherein the agent is an antibody that binds to CD63 and inhibits degranulation at a concentration of 10 μg/ml or less.

6. A method of treating an allergic condition in a mammal comprising administering to the mammal an effective amount of an antibody that binds to CD63.

7. The method of claim 6 wherein the allergic condition is selected from the group consisting of asthma, rhinitis, hay fever, conjunctivitis, atopic dermatitis, atopic eczema, passive cutaneous anaphylaxis, systemic allergic reactions and intestinal reactions of the gastrointestinal system.

8. The method of claim 6 wherein the allergic condition is an autoimmune disease.

9. The method of claim 6 wherein the mammal is a human.

10. The antibody of claim 1 wherein the antibody inhibits degranulation at a concentration of 10 μg/ml or less.

11. The method of claim 8 wherein the autoimmune disease is selected from the group consisting of multiple sclerosis, rheumatoid arthritis and mastocytosis.

12. The method of claim 8 wherein the autoimmune disease is mastocytosis.

13. A method of treating a neoplastic disease in a subject comprising administering to the subject an effective amount of an antibody that binds to CD63

14. The method of claim 13 wherein the neoplastic disease is a CD63-expressing lymphoma and/or CD63-expressing leukemia.

15. The method of claim 13 wherein the subject is a human.

16. The method of claim 13 wherein the antibody inhibits degranulation at a concentration of 10 μg/ml or less.

17. The antibody of claim 1 wherein the antibody is an antibody fragment.

18. The antibody of claim 17 wherein the antibody fragment is selected from the group consisting of an Fv fragment, an Fab fragment, an Fab′ fragment and an F(ab′)₂ fragment.

19. The method of claim 6 wherein the antibody is an antibody fragment.

20. The method of claim 13 wherein the antibody is an antibody fragment.


SEQUENCE LISTING (4 sequences)

SEQ ID NO: 1 - DNA, 925 bp, Homo sapiens
ggcacgaggggccggggggcgcagctagagagccccggagccgcggcgggagaggaacgcgcagccagccttgggaagcccaggcccggcagccatggcggtggaaggaggaatgaaatgtgtgaagttcttgctctacgtcctcctgctggccttttgcgcctgtgcagtgggactgattgccgtgggtgtcggggcacagcttgtcctgagtcagaccataatccagggggctacccctggctctctgttgccagtggtcatcatcgcagtgggtgtcttcctcttcctggtggcttttgtgggctgctgcggggcctgcaaggagaactattgtcttatgatcacgtttgccatctttctgtctcttatcatgttggtggaggtggccgcagccattgctggctatgtgtttagagataaggtgatgtcagagtttaataacaacttccggcagcagatggagaattacccgaaaaacaaccacactgcttcgatcctggacaggatgcaggcagattttaagtgctgtggggctgctaactacacagattgggagaaaatcccttccatgtcgaagaaccgagtccccgactcctgctgcattaatgttactgtgggctgtgggattaatttcaacgagaaggcgatccataaggagggctgtgtggagaagattgggggctggctgaggaaaaatgtgctggtggtagctgcagcagcccttggaattgcttttgtcgaggttttgggaattgtctttgcctgctgcctcgtgaagagtatcagaagtggctacgaggtgatgtaggggtctggtctcctcagcctcctcatctgggggagtggaatagtatcctccaggtttttcaattaaacggattattttttcagaccgaaaagaaaaaaaaaaaaaaaaaaaaaa

SEQ ID NO: 2 - protein, 238 aa, Homo sapiens
MAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVM

SEQ ID NO: 3 - DNA, 68 bp, Artificial Sequence
  oligonucleotide primer
cagaattcccaccatgggcgactacaaggacgacgatgacaaggcggtggaaggaggaatgaagtgtg

SEQ ID NO: 4 - DNA, 35 bp, Artificial Sequence
  oligonucleotide primer
cacaagcttgggctacattacttcgtagccactcc